(19) United States
(12) Patent Application Publication
(10) Pub. No.: US 2015/0290473 A1
(43) Pub. Date: Oct. 15, 2015

(54) Radiotherapy system

(71) Applicant: Elekta AB (publ), Stockholm (SE)

(72) Inventors: Yaroub Taha Abdul-Hamid, Haywards Heath (GB); Chris Bean, Crawley (GB)

(73) Assignee: Elekta AB (publ), Stockholm (SE)

(21) Appl. No.: 14/250,771

(22) Filed: Apr. 11, 2014

(51) Int. Cl. A61N 5/10

(52) U.S. Cl. CPC A61N 5/1045; A61N 2005/1095

(57) ABSTRACT

A radiotherapy system comprising a source of radiation, for generating a beam of therapeutic radiation; a field light, for generating a beam of optical light that emulates the beam of therapeutic radiation; and a collimating device, for collimating the beam of therapeutic radiation and the beam of optical light. The field light comprises a single light-emitting diode (LED) having a single die, and wherein the radiotherapy system does not comprise any lenses outside the LED through which the beam of optical light passes.

### TECHNICAL FIELD

The present invention relates to radiotherapy systems, and particularly devices and methods for illuminating a field of treatment.

### BACKGROUND

Radiotherapy involves the action of highly energized ionizing radiation to damage or destroy unhealthy cells within a patient. The radiation is generally collimated and/or focussed into a beam, and directed towards a target within the patient from an offset location.

The radiation is damaging to all cells—healthy and unhealthy alike—on which it impinges. Healthy tissue adjacent to the target will inevitably receive some dose. For example, the target will generally be located inside the patient, and thus some dose will be deposited in the healthy tissue in front of and behind the target (from the beam's eye view). Further, the target will rarely have a regular geometric cross-section and thus is unlikely to conform precisely to the shape of the beam.

The former cause of dose in healthy tissue can be mitigated by rotating the source of radiation around the patient. By keeping the radiation beam directed towards the target throughout rotation, the radiation dose in the target is maximized, while dose within the surrounding healthy tissue is reduced.

The latter cause of dose in healthy tissue can be mitigated by collimating the beam to conform accurately to the cross-section of the target or some other clinically useful shape. When the source of radiation is rotated around the patient as described above, this can involve shaping the radiation beam differently at different angles of rotation.

Various devices for collimating a beam in this manner are known in the art. One example is the multi-leaf collimator, in which two banks of laterally spaced, thin leaves are arranged on opposite sides of a radiation window. Each leaf is individually controllable to move longitudinally into and out of the radiation window so as to block that part of the radiation beam. The combined action of all leaves results in a radiation beam which can take any arbitrary shape as desired.

As part of the process of setting up and evaluating a particular treatment plan, it is known to provide radiotherapy systems with a field light, that is, an optical light source which replicates the source of radiation and thus allows the action of the collimator to be visually checked. The field light needs to have a number of properties in order to accurately fulfil its task. First, it needs to be bright enough to be clearly seen in an illuminated treatment room environment. Second, as the field light is emulating a radiation source which is approximately a point source, the field light too should ideally have as small a source point as possible. The larger the size of the source, the more diffuse the generated beam will be, increasing the penumbra of the light field and reducing its accuracy as a tool for verifying collimator performance.

Current field lights employ a halogen projector bulb as the light source. A lens is used to focus the light through a small aperture in order to replicate the smaller radiation source and to provide sufficient luminescence that the optical beam can be seen.

The lifetime of the halogen bulb is such that it needs to be routinely replaced, multiple times a year. Each time the bulb is replaced, a number of other components will typically have to be removed in order to access the bulb housing. This means that, once the bulb is replaced, further time is spent ensuring the system is set up correctly.

### SUMMARY OF INVENTION

According to a first aspect of the present invention, there is provided a radiotherapy system comprising a source of radiation, for generating a beam of therapeutic radiation; a field light, for generating a beam of optical light that emulates the beam of therapeutic radiation; and a collimating device, for collimating the beam of therapeutic radiation and the beam of optical light. The field light comprises a single light-emitting diode (LED) having a single die, and wherein the radiotherapy system does not comprise any lenses outside the LED through which the beam of optical light passes.

### BRIEF DESCRIPTION OF THE DRAWINGS

For a better understanding of the present invention, and to show more clearly how it may be carried into effect, reference will now be made, by way of example, to the following drawings, in which:

FIG. 1 shows a radiotherapy system according to embodiments of the present invention; and

FIG. 2 shows a field light according to embodiments of the invention.

### DETAILED DESCRIPTION

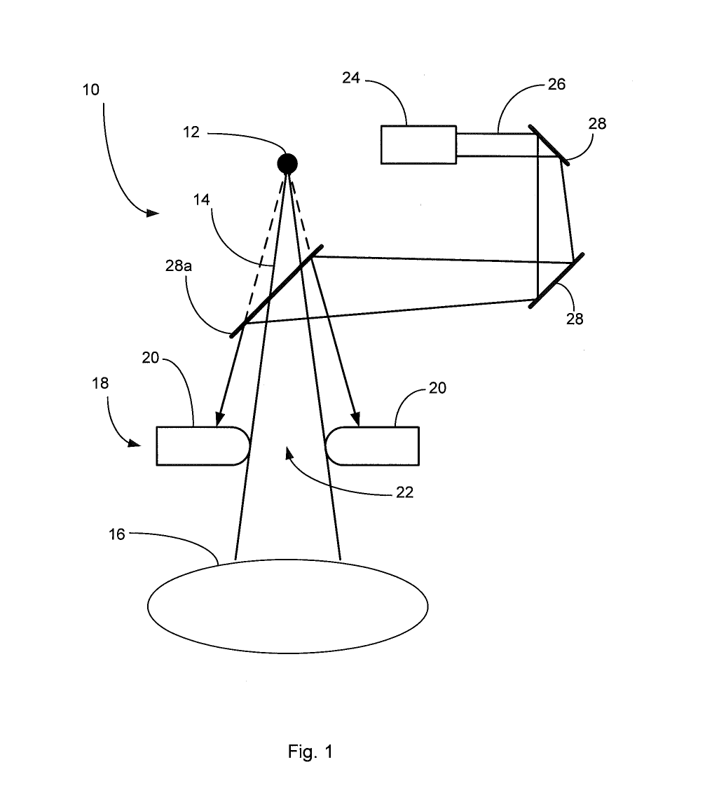

FIG. 1 shows a radiotherapy system 10 according to embodiments of the present invention. As will be clear to those skilled in the art, many features inessential to description of the invention are not shown for the purposes of clarity.

The system comprises a source of therapeutic radiation 12. The radiation may be any ionizing radiation known in the art, such as protons, electrons or x-rays, and the source may be any device or substance known to produce that radiation. For example, the source may comprise a linear accelerator, or a radioactive material such as cobalt-60. In order to have a therapeutic effect, the radiation will typically have an energy in the megavoltage range. The source 12 generates a beam of therapeutic radiation 14, and this is directed towards a patient 16 lying on a support. One or more primary collimators (not illustrated) can be used to collimate the beam 14 into the desired overall shape, such as a cone beam, fan beam, or any other shape known in the art. One or more secondary collimators 18 are provided to shape the beam as desired to conform to a particular desired shape for treating the patient 16.

The secondary collimator 18 comprises one or more collimating elements 20, which are controllable individually or in unison to move across a radiation window 22. When the radiation beam 14 is directed through that window 22, the collimating elements 20 act to block the beam in part and so collimate the beam to conform to a desired shape (i.e. the unblocked parts of the window 22).

The secondary collimator 18 may take any form known in the art, such as block collimators or leaf collimators. In one embodiment, however, the secondary collimator 18 is a multi-leaf collimator, and the collimating elements 20 are the leaves of the multi-leaf collimator.

Multi-leaf collimators comprise one or more banks of laterally spaced elongate leaves. In the illustrated embodiment, the collimator 18 comprises two banks of leaves arranged on opposite sides of the window 22. Each leaf is individually controllable to move longitudinally into and out of the radiation window 22. The leaves are relatively deep in the direction of the radiation beam, and are typically manufactured from a dense material with high atomic number (such as tungsten), and thus each leaf is effective in blocking the highly energized radiation from reaching the patient 16. The combination of all leaves acts to define an arbitrarily shaped aperture within the radiation window 22, thus collimating the radiation beam 14 to that shape.

In order to check the correct functioning of the secondary collimator 18, the radiotherapy system further comprises a field light 24. This generates a beam of optical light 26 which can be used to replicate the radiation beam 14. One or more optical devices 28 (e.g. mirrors) are provided to direct the optical beam 26 through the secondary collimator 18, along the same optical path as the radiation beam 14. In particular, at least one optical device 28a is provided in the path of the radiation beam 14 (between the radiation source 12 and the secondary collimator 18) and in the path of the optical beam 26 (between the field light 24 and the secondary collimator 18). Were both beams 14, 26 active at the same time, the beams would be congruent from the optical device 28a, through the secondary collimator 18, to the patient. The optical beam 26 is thus subject to the same collimating effect as the radiation beam 14, and projected onto the patient 16 (or another object in place of the patient) is an optical light field corresponding to the shape of the radiation beam 14. The optical beam can thus be used to check the correct operation of the secondary collimator 18, i.e. that each collimating element 20 is moving to a desired position in order to collimate the radiation, without generating the radiation beam 14.

The optical beam 26 is thus intended to closely replicate the radiation beam 14, and in order to achieve that, the field light 24 must resemble the source of radiation 12 as closely as possible. The field light is thus placed at a location and the optical elements 28 arranged such that the optical beam 26 emanates from the same virtual location relative to the collimator 18 as the radiation source 12. The optical light source within the field light 24 should also closely resemble the source of radiation 12.

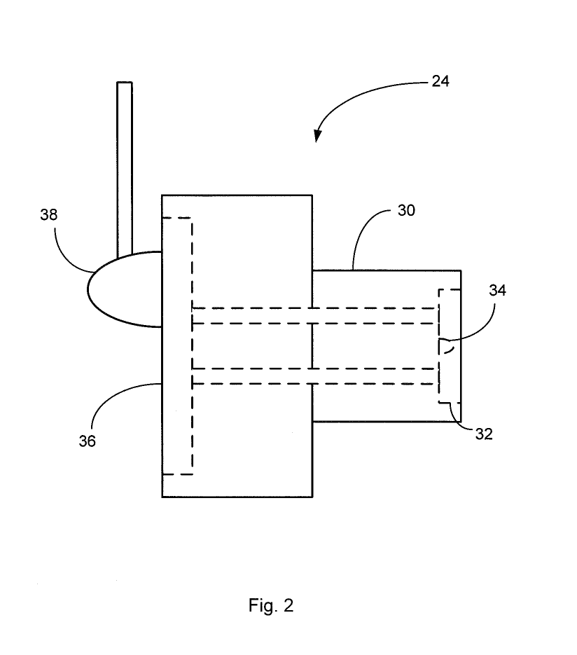

FIG. 2 shows a field light 24 according to embodiments of the invention.

The field light 24 comprises a housing 30, a mount 32 coupled to the housing, a light source 34 provided on the mount 32, a power control board 36 for controlling the power to the light source 34, and a power connector 38 for coupling the board 36 to a source of power (not illustrated).

In embodiments of the invention, the light source 34 comprises a light-emitting diode (LED). In further embodiments of the invention, the light source 34 comprises a single LED. In one embodiment, the LED may have a cross-sectional area less than 4.9 mm² In another embodiment, the LED may have a cross-sectional area less than 2.5 mm². In a further embodiment, the LED may have a cross-sectional area less than 1.5 mm². The LED may be positioned and have a luminosity such that the optical beam 26 has an average of at least 25 lux at the location of the patient 16 (e.g. at the isocentre of the system 10).

In embodiments of the invention, the LED comprises a single die. That is, it comprises a single semiconductor die from which photons emanate. Those skilled in the art will appreciate that the LED may also comprise a reflective cavity behind the semiconductor die, and a package which may have a lens cover. The LED 34 is thus small and bright enough that it can generate an optical beam 26 having the required properties on its own, without the aid of additional lenses (i.e. lenses outside the LED, not including the LED cover) or an aperture through which conventional optical beams must pass. In fact, it is found that when used with additional lenses, the intensity of the optical beam 26 is reduced. Thus not only does the field light 24 not require lenses to intensify the optical beam 26, the presence of lenses actively reduces the beam's brightness. The field light 24 as a whole has fewer parts and requires less set up. Further, the LED has a greater lifetime than existing halogen lamps, and so requires replacement less frequently (with the associated set up and recalibration). In fact, the LED may not need replacing over the course of the radiotherapy system's lifetime.

Embodiments of the present invention thus provide a radiotherapy system, and particularly a field light for a radiotherapy system, which is simpler and cheaper to manufacture and run, and requires less frequent maintenance than existing systems.

Those skilled in the art will appreciate that various amendments and alterations can be made to the embodiments described above without departing from the scope of the invention as defined in the claims appended hereto.

For example, the illustrated embodiments show an optical device 28a in the path of the optical and therapeutic beams 26, 14, designed to ensure the beams emanate from substantially the same virtual source. In alternative arrangements, the field light 24 and radiation source 12 may be provided on a movable gantry, such that the field light 24 and radiation source 12 can be interchanged into the same position suitable for generating a beam passing through the secondary collimator 18. Alternative solutions will be apparent to the skilled person, without departing from the scope of the claims.

1. A radiotherapy system comprising:
a source of radiation, for generating a beam of therapeutic radiation;
a field light, for generating a beam of optical light that emulates the beam of therapeutic radiation; and
a collimating device, for collimating the beam of therapeutic radiation and the beam of optical light;
wherein the field light comprises a single light-emitting diode (LED) having a single die, and wherein the radiotherapy system does not comprise any lenses outside the LED through which the beam of optical light passes.

2. The radiotherapy system according to claim 1, wherein the LED has a cross-sectional area of less than 4.9 mm².

3. The radiotherapy system according to claim 1, wherein the LED has a cross-sectional area of less than 2.5 mm².

4. The radiotherapy system according to claim 1, wherein the LED has a cross-sectional area of less than 1.5 mm².

5. The radiotherapy system according to claim 1, wherein the beam of optical light has a luminosity of at least 25 lux at an isocentre of the radiotherapy system.

6. The radiotherapy system according to claim 1, wherein the beam of therapeutic radiation and the beam of optical light emanate from the same virtual location relative to the collimating device.

7. The radiotherapy system according to claim 1, wherein the beam of optical light and the beam of therapeutic radiation are congruent as the beams pass through the collimating device.

8. The radiotherapy system according to claim 1, wherein the collimating device comprises one or more of: a multi-leaf collimator and a block collimator.
